SEQUENCE LISTING (63 sequences)

SEQ ID NO: 1 - RNA, 21 nt, Homo sapiens
  source 1..21: mol_type = other RNA
gtgcattgtagttgcattgca

SEQ ID NO: 2 - RNA, 20 nt, Homo sapiens
  source 1..20: mol_type = other RNA
gtgcattgctgttgcattgc

SEQ ID NO: 3 - RNA, 22 nt, Homo sapiens
  source 1..22: mol_type = other RNA
tggagtgtgacaatggtgtttg

SEQ ID NO: 4 - RNA, 22 nt, Homo sapiens
  source 1..22: mol_type = other RNA
gcctgctggggtggaacctggt

SEQ ID NO: 5 - RNA, 21 nt, synthetic construct
  source 1..21: mol_type = other RNA
tgcaatgcaactacaatgcac

SEQ ID NO: 6 - RNA, 23 nt, synthetic construct
  source 1..23: mol_type = other RNA
acaaacaccattgtcacactcca

SEQ ID NO: 7 - DNA, 20 bp, synthetic construct
  source 1..20: mol_type = other DNA
cagagctgcacggaaacatg

SEQ ID NO: 8 - DNA, 20 bp, synthetic construct
  source 1..20: mol_type = other DNA
tcatcagcgagcatcaggtc

SEQ ID NO: 9 - DNA, 20 bp, synthetic construct
  source 1..20: mol_type = other DNA
agccatgtacgtagccatcc

SEQ ID NO: 10 - DNA, 20 bp, synthetic construct
  source 1..20: mol_type = other DNA
ctctcagctgtggtggtgaa

SEQ ID NO: 11 - RNA, 22 nt, synthetic construct
  source 1..22: mol_type = other RNA
accatattgcgcgtatagtcgc

SEQ ID NO: 12 - DNA, 20 bp, synthetic construct
  source 1..20: mol_type = other DNA
aggtccctgtcatgcttctg

SEQ ID NO: 13 - DNA, 20 bp, synthetic construct
  source 1..20: mol_type = other DNA
tctggacccattccttcttg

SEQ ID NO: 14 - DNA, 22 bp, synthetic construct
  source 1..22: mol_type = other DNA
actaccaagccagtgctgcgaa

SEQ ID NO: 15 - DNA, 22 bp, synthetic construct
  source 1..22: mol_type = other DNA
atcacaggcgaagtccaatccg

SEQ ID NO: 16 - DNA, 20 bp, synthetic construct
  source 1..20: mol_type = other DNA
cgtcagccgatttgctatct

SEQ ID NO: 17 - DNA, 20 bp, synthetic construct
  source 1..20: mol_type = other DNA
cggactccgcaaagtctaag

SEQ ID NO: 18 - DNA, 20 bp, synthetic construct
  source 1..20: mol_type = other DNA
attcaaggctgtgggaaatg

SEQ ID NO: 19 - DNA, 20 bp, synthetic construct
  source 1..20: mol_type = other DNA
ctcagagtacgccagggaac

SEQ ID NO: 20 - DNA, 22 bp, synthetic construct
  source 1..22: mol_type = other DNA
caccttcctctggcacagttac

SEQ ID NO: 21 - DNA, 20 bp, synthetic construct
  source 1..20: mol_type = other DNA
tcccttcctcgatgccactt

SEQ ID NO: 22 - DNA, 20 bp, synthetic construct
  source 1..20: mol_type = other DNA
ttgcttcagctccacagaga

SEQ ID NO: 23 - DNA, 20 bp, synthetic construct
  source 1..20: mol_type = other DNA
tggttgtagagggcaaggac

SEQ ID NO: 24 - DNA, 23 bp, synthetic construct
  source 1..23: mol_type = other DNA
cgtgacagtagataccaacgctc

SEQ ID NO: 25 - DNA, 22 bp, synthetic construct
  source 1..22: mol_type = other DNA
catagcactcggcatcttctcc

SEQ ID NO: 26 - DNA, 22 bp, synthetic construct
  source 1..22: mol_type = other DNA
gcttcgtgttcaccaaggagga

SEQ ID NO: 27 - DNA, 22 bp, synthetic construct
  source 1..22: mol_type = other DNA
gtcctagaccagtgttctgagc

SEQ ID NO: 28 - RNA, 23 nt, Homo sapiens
  source 1..23: mol_type = other RNA
aggcagtgtagttagctgattgc

SEQ ID NO: 29 - RNA, 25 nt, Homo sapiens
  source 1..25: mol_type = other RNA
tccctgaggagccctttgagcctga

SEQ ID NO: 30 - RNA, 19 nt, Homo sapiens
  source 1..19: mol_type = other RNA
actggacttggaggcagaa

SEQ ID NO: 31 - RNA, 22 nt, Homo sapiens
  source 1..22: mol_type = other RNA
caaagaattctccttttgggct

SEQ ID NO: 32 - RNA, 22 nt, Homo sapiens
  source 1..22: mol_type = other RNA
tggcagtgtcttagctggttgt

SEQ ID NO: 33 - RNA, 22 nt, Homo sapiens
  source 1..22: mol_type = other RNA
tccctgagaccctaacttgtga

SEQ ID NO: 34 - RNA, 22 nt, Homo sapiens
  source 1..22: mol_type = other RNA
aaacaaacatggtgcacttctt

SEQ ID NO: 35 - RNA, 22 nt, Homo sapiens
  source 1..22: mol_type = other RNA
actccagccccacagcctcagc

SEQ ID NO: 36 - RNA, 22 nt, Homo sapiens
  source 1..22: mol_type = other RNA
cattgcacttgtctcggtctga

SEQ ID NO: 37 - RNA, 22 nt, Homo sapiens
  source 1..22: mol_type = other RNA
tccagcatcagtgattttgttg

SEQ ID NO: 38 - RNA, 22 nt, Homo sapiens
  source 1..22: mol_type = other RNA
tattgcacttgtcccggcctgt

SEQ ID NO: 39 - RNA, 20 nt, Homo sapiens
  source 1..20: mol_type = other RNA
cggcggggctcggagccggg

SEQ ID NO: 40 - RNA, 22 nt, Homo sapiens
  source 1..22: mol_type = other RNA
tggcgggggtagagctggctgc

SEQ ID NO: 41 - RNA, 21 nt, Homo sapiens
  source 1..21: mol_type = other RNA
cggcggggacggcgattggtc

SEQ ID NO: 42 - RNA, 22 nt, Homo sapiens
  source 1..22: mol_type = other RNA
aggcggggcgccgcgggaccgc

SEQ ID NO: 43 - RNA, 22 nt, Homo sapiens
  source 1..22: mol_type = other RNA
tggtgggcacagaatctggact

SEQ ID NO: 44 - RNA, 22 nt, Homo sapiens
  source 1..22: mol_type = other RNA
tggtgggccgcagaacatgtgc

SEQ ID NO: 45 - RNA, 20 nt, synthetic construct
  source 1..20: mol_type = other RNA
gcaatgcaacagcaatgcac

SEQ ID NO: 46 - RNA, 22 nt, synthetic construct
  source 1..22: mol_type = other RNA
accaggttccaccccagcaggc

SEQ ID NO: 47 - RNA, 23 nt, synthetic construct
  source 1..23: mol_type = other RNA
gcaatcagctaactacactgcct

SEQ ID NO: 48 - RNA, 25 nt, synthetic construct
  source 1..25: mol_type = other RNA
tcaggctcaaagggctcctcaggga

SEQ ID NO: 49 - RNA, 19 nt, synthetic construct
  source 1..19: mol_type = other RNA
ttctgcctccaagtccagt

SEQ ID NO: 50 - RNA, 22 nt, synthetic construct
  source 1..22: mol_type = other RNA
agcccaaaaggagaattctttg

SEQ ID NO: 51 - RNA, 22 nt, synthetic construct
  source 1..22: mol_type = other RNA
acaaccagctaagacactgcca

SEQ ID NO: 52 - RNA, 22 nt, synthetic construct
  source 1..22: mol_type = other RNA
tcacaagttagggtctcaggga

SEQ ID NO: 53 - RNA, 22 nt, synthetic construct
  source 1..22: mol_type = other RNA
aagaagtgcaccatgtttgttt

SEQ ID NO: 54 - RNA, 22 nt, synthetic construct
  source 1..22: mol_type = other RNA
gctgaggctgtggggctggagt

SEQ ID NO: 55 - RNA, 22 nt, synthetic construct
  source 1..22: mol_type = other RNA
tcagaccgagacaagtgcaatg

SEQ ID NO: 56 - RNA, 22 nt, synthetic construct
  source 1..22: mol_type = other RNA
caacaaaatcactgatgctgga

SEQ ID NO: 57 - RNA, 22 nt, synthetic construct
  source 1..22: mol_type = other RNA
acaggccgggacaagtgcaata

SEQ ID NO: 58 - RNA, 20 nt, synthetic construct
  source 1..20: mol_type = other RNA
cccggctccgagccccgccg

SEQ ID NO: 59 - RNA, 22 nt, synthetic construct
  source 1..22: mol_type = other RNA
gcagccagctctacccccgcca

SEQ ID NO: 60 - RNA, 21 nt, synthetic construct
  source 1..21: mol_type = other RNA
gaccaatcgccgtccccgccg

SEQ ID NO: 61 - RNA, 22 nt, synthetic construct
  source 1..22: mol_type = other RNA
gcggtcccgcggcgccccgcct

SEQ ID NO: 62 - RNA, 22 nt, synthetic construct
  source 1..22: mol_type = other RNA
agtccagattctgtgcccacca

SEQ ID NO: 63 - RNA, 22 nt, synthetic construct
  source 1..22: mol_type = other RNA
gcacatgttctgcggcccacca

(19) United States
(12) Patent Application Publication
(10) Pub. No.: US 2026/0242785 A1
(43) Pub. Date: Aug. 20, 2026

(54) A COMBINATION OF ANTIMIR OLIGONUCLEOTIDES FOR TREATING A DISEASE

(71) Applicant: SIRT6 LTD, Ness Ziona (IL)

(72) Inventors: Haim COHEN, Modiin-Maccabim-Reut (IL); Hagit ASHUSH, Ashkelon (IL); Yaki EIDELSTEIN, Rehovot (IL); Boaz MISHOLI, Tel Aviv (IL); Reuven GIL, Bat-Yam (IL)

(73) Assignee: SIRT6 LTD, Ness Ziona (IL)

(21) Appl. No.: 18/993,236

(22) PCT Filed: Jul. 13, 2023
(86) PCT No.: PCT/IL2023/050736

§ 371 Date: Jan. 10, 2025

Related U.S. Application Data
(60) Provisional application No. 63/368,294, filed on Jul. 13, 2022.

(51) Int. Cl. C12N 15/113; A61P 1/16

(52) U.S. Cl. CPC C12N 15/113; A61P 1/16; C12N 2310/113; C12N 2310/321; C12N 2310/322; C12N 2310/343; C12N 2310/3515; C12N 2320/31

(57) ABSTRACT

Provided are methods for treating a disease or condition in a subject in need thereof by partially inhibiting the silencing activity of at least two SIRT6-targeted miRNAs in said subject, thereby increasing SIRT6 protein levels. The partial inhibition of the silencing activity is achieved by administering a combination of Anti-miRNAs, preferably modified Anti-miRNAs.

### TECHNOLOGICAL FIELD

The present disclosure concerns therapeutic methods based on increasing the expression of a target protein, particularly SIRT6 expression.

### BACKGROUND ART

References considered to be relevant as background to the presently disclosed subject matter are listed below:
1. Chang, AR. et al SIRT6, a Mammalian Deacylase with Multitasking Abilities. Physiol Rev 100:145-169, 2020.
2. Finkel, T. et al Recent progress in the biology and physiology of sirtuins. Nature 460:587-591, 2009.
3. Baer, C. et al Genome-wide epigenetic regulation of miRNAs in cancer. Cancer Res. 73:473-7 (2013).
4. Li, N. et al. Downregulation of SIRT6 by miR-34c-5p is associated with poor prognosis and promotes colon cancer proliferation through inhibiting apoptosis via the JAK2/STAT3 signaling pathway. International Journal of Oncology 52 (5): 1515-1527 (2018).
5. Hu, Y. et al. MicroRNA-351-5p aggravates intestinal ischaemia/reperfusion injury through the targeting of MAPK13 and Sirtuin-6. British Journal of Pharmacology 175, 3594-3609 (2018).
6. Joo, D. et al. MicroRNA-378b regulates α-1-type 1 collagen expression via sirtuin 6 interference. Molecular Medicine Reports 16, 8520-8524 (2017).
7. Ruan, L., Chen, J., Ruan, L., Yang, T. & Wang, P. MicroRNA-186 suppresses lung cancer progression by targeting SIRT6. Cancer Biomarkers 21, 415-423 (2018).
8. Ruan, L. et al miR-34a inhibits tumorigenesis of NSCLC via targeting SIRT6. Int J Clin Exp Pathol. 11, 1135-1145 (2018)
9. Song, S. MiR-125b attenuates human hepatocellular carcinoma malignancy through targeting SIRT6. Am J Cancer Res. 8, 993-1007 (2018).
10. Shi, M. et al. Statin suppresses sirtuin 6 through miR-495, increasing Fox01-dependent hepatic gluconeogenesis. Theranostics 10, 11416-11427 (2020).
11. Sharma, A. et al. The Role of SIRT6 Protein in Aging and Reprogramming of Human Induced Pluripotent Stem Cells. Journal of Biological Chemistry 288, 18439-18447 (2013).
12. Wang, S. et al. Transfer of microRNA-25 by colorectal cancer cell-derived extracellular vesicles facilitates colorectal cancer development and metastasis. Molecular Therapy—Nucleic Acids Vol. 23, 552-564 (2021).
13. Jiang, H. et al. MicroRNA-338-3p as A Novel Therapeutic Target for Intervertebral Disc Degeneration. (2020). doi: 10.21203/rs.3.rs-101234/v1
14. Chiba et al., PLOS ONE 11 (10), 2016 DOI: 10.1371/journal.pone.0164191.

Acknowledgement of the above references herein is not to be inferred as meaning that these are in any way relevant to the patentability of the presently disclosed subject matter.

### BACKGROUND

Mammalian sirtuins have emerged in recent years as critical modulators of multiple biological processes, regulating cellular metabolism, DNA repair, gene expression, and mitochondrial biology. As such, they evolved to play key roles in organismal homeostasis, and defects in these proteins have been linked to a plethora of diseases, including cancer, neurodegeneration, and aging [1].

Thus far, scientists identified seven mammalian homologues, named SIRT1 to SIRT7, or sirtuins (Sir2-like, since all are homologues of the yeast Sir2 enzyme) [2], among them SIRT6.

SIRT6 is a chromatin deacylase which is found exclusively in the cell nucleus.

Among others, SIRT6 was found to be involved in glucose homeostasis, DNA Repair, telomeric function, cellular differentiation, mitosis, meiosis, as well as cancer [1].

MicroRNAs (also referred to herein as miRNAs or miRs) are a class of short naturally occurring non-coding RNA molecules (17-25 nucleotides), which have a critical role in the negative regulation of their target genes. miRs regulate gene expression in the human genome via sequence specific binding to the 3′-untranslated regions (3′-UTR) of their target mRNA [3]. As a result of this binding, the miRs block translation or cause degradation of their target messenger RNAs.

Several microRNAs were shown to be associated with downregulation of SIRT6, including for example 34c-5p [4], 351-5p [5], 378b [6], 186 [7], 34a [8], 125b [9], 495 [10], 766 [11], 25 [12], 338-3p [13].

WO 2014/118272 discloses conjugated oligonucleotides which target microRNA-122 and comprise an antisense oligomer which is complementary to the microRNA-122 target sequence wherein the oligomer is conjugated to an asialoglycoprotein receptor targeting moiety.

U.S. Pat. No. 9,241,950 discloses miR-33 inhibitors and uses thereof to decrease inflammation. The miR-33 inhibitor is preferably an antagomir having a single-stranded nucleic acid sequence that is complementary to at least 12 contiguous nucleotides in miR-33 and form a duplex with miR-33 under physiological conditions.

U.S. Pat. No. 8,809,294 discloses a method of inhibiting the activity, function, or amount of miR-33, using a modified oligonucleotide which is complementary to miR-33 and consists of several linked monomeric subunits each comprising a modified sugar moiety.

U.S. Pat. Nos. 9,422,561, 9,650,637, and 10,036,023 disclose methods for silencing of miRs which down regulate the expression of the sirtuin-6 (SIRT6) gene (i.e., miR-33A, miR-33B, miR-122, and miR-370) using single anti-miR agents, thereby increasing SIRT6 expression levels.

### GENERAL DESCRIPTION

In a first of its aspects, the present invention provides a method for treating a disease or condition in a subject in need thereof comprising a step of partially inhibiting the silencing activity of at least two SIRT6-targeted miRNAs in said subject, thereby increasing SIRT6 protein levels.

In one embodiment, said method comprises administering to said subject a therapeutically effective amount of at least two antisense oligonucleotides which target said at least two SIRT6-targeted miRNAs.

In another aspect, the present invention provides an antisense oligonucleotide which targets a first sirtuin-6 (SIRT6)-targeted miRNA for use in combination with at least one additional antisense oligonucleotide which targets at least one other SIRT6-targeted miRNA in a method of treatment of a disease or condition in a subject, wherein said antisense oligonucleotides cause partial inhibition of the silencing activity of said SIRT6-targeted miRNAs.

In another aspect, the present invention provides a pharmaceutical composition comprising a combination of at least two antisense oligonucleotides which target at least two SIRT6-targeted miRNAs and a suitable carrier or excipient for use in a method of treatment of a disease, wherein said antisense oligonucleotides cause partial inhibition of the silencing activity of said at least two SIRT6-targeted miRNAs.

In one embodiment, said amount, or therapeutically effective amount is an amount sufficient to increase SIRT6 mRNA level or SIRT6 protein level while affecting to a lesser degree the expression of other genes.

In one embodiment, said at least two antisense oligonucleotides and said effective amount of each of said antisense oligonucleotides are selected to specifically suit said subject's disease or condition.

In one embodiment, said method further comprises measuring the levels of SIRT6 mRNA expression or the levels of SIRT6 protein in said subject, prior to and/or during treatment, wherein the types and amounts of said antisense oligonucleotides are adjusted according to the measured levels of SIRT6.

In one embodiment, said disease or condition is selected from a group consisting of cancer (e.g., colorectal cancer, lung cancer, hepatocellular carcinoma), ischemic tissue damage (e.g., intestinal ischemia-reperfusion (II/R) injury), short bowel syndrome, degenerative bone diseases (e.g., intervertebral disc degeneration (IDD)), shock), liver diseases (e.g., non-alcoholic fatty liver disease (NAFLD), NASH, fibrosis, hepatitis), frailty syndrome, Alzheimer's disease, atherosclerosis, lung fibrosis, and dermal conditions (e.g. collagen depletion, UV damage).

In one embodiment, said subject is a human subject or an animal subject.

In one embodiment, said at least two SIRT6-targeted miRNA are selected from the group consisting of miR-33A, miR-33B, miR-122, miR-370, miR-34c-5p, miR-351-5p, miR-378b, miR-186, miR-34a, miR-125b, miR-495, miR-766, miR-25, miR-338-3p, miR-92a-3p, miR-10396b-5p, miR-6787-5p, miR-1908-5p, miR-663a, miR-541-3p, and miR-654-5p.

In one embodiment, said at least two SIRT6-targeted miRNAs are selected from the group consisting of the miRNAs denoted by SEQ ID Nos: 1-4 and 28-44.

In one embodiment, said at least two, or said first and said at least one other, SIRT6-targeted miRNA is miR-33A and miR-122.

In one embodiment, said at least two antisense oligonucleotides comprise anti-miRs selected from the group consisting of anti-miR-33A, anti-miR-33B, anti-miR-122, anti-miR-370, anti-miR-34c-5p, anti-miR-351-5p, anti-miR-378b, anti-miR-186, anti-miR-34a, anti-miR-125b, anti-miR-495, anti-miR-766, anti-miR-25, anti-miR-338-3p, anti-miR-92a-3p, anti-miR-10396b-5p, anti-miR-6787-5p, anti-miR-1908-5p, anti-miR-663a, anti-miR-541-3p, and anti-miR-654-5p.

In one embodiment, said at least two antisense oligonucleotides comprise anti-miRs selected from the group consisting of the anti-miRs denoted by SEQ ID Nos: 5, 6 and 45-63.

In one embodiment, said at least two antisense oligonucleotides comprise SEQ ID NO: 5 and SEQ ID NO: 6 or an active fragment thereof.

In one embodiment, said antisense oligonucleotides are conjugated to a moiety.

In one embodiment said moiety is a targeting moiety.

In one embodiment said moiety is selected from a group consisting of N-acetylgalactosamine (GalNAc), cholesterol and vitamin E.

In one embodiment said antisense oligonucleotides comprise chemically modified nucleic acids.

In one embodiment said modified nucleic acids comprise 2′ O-methoxyethyl-RNA (MOE) or 2′-F modifications.

In one embodiment said anti-miR-33 comprises the modified sequence:
TmGmCfAfAfTfGfCfAfAfCmTmAmCfAfAfTfGfCfAmCm,
and said anti-miR-122 comprises the modified sequence:
AmCmAfAfAfCfAfCfCfAfTmTmGmUfCfAfCfAfCfUfCfCmAm
wherein m is a 2′ MOE modified base and fis a 2′F modified base.

In a specific embodiment, the present invention provides a method for treating non-alcoholic fatty liver disease (NAFLD), NASH, or liver fibrosis in a subject in need thereof comprising administering to said subject an antisense oligonucleotide comprising anti-miR-33A as denoted by SEQ ID NO: 5 and an antisense oligonucleotide comprising anti-miR-122 as denoted by SEQ ID NO: 6 or a pharmaceutical composition comprising said antisense oligonucleotides.

In another specific embodiment, the present invention provides an antisense oligonucleotide comprising anti-miR-33A as denoted by SEQ ID NO: 5 for use in combination with an antisense oligonucleotide comprising anti-miR-122 as denoted by SEQ ID NO: 6 in a method for treating non-alcoholic fatty liver disease (NAFLD), NASH, or liver fibrosis in a subject in need thereof.

In another specific embodiment, the present invention provides a pharmaceutical composition comprising an antisense oligonucleotide comprising anti-miR-33A as denoted by SEQ ID NO: 5 and an antisense oligonucleotide comprising anti-miR-122 as denoted by SEQ ID NO: 6 for use in a method for treating non-alcoholic fatty liver disease (NAFLD), NASH, or liver fibrosis in a subject in need thereof.

In another aspect, the present invention provides a method for increasing SIRT6 mRNA level or SIRT6 protein level in a cell comprising contacting said cell with an effective amount of at least two antisense oligonucleotides which target at least two SIRT6-targeted miRNAs, and which partially inhibit the silencing activity of said at least two SIRT6-targeted miRNAs.

In another aspect, the present invention provides a method for increasing the amount of a target protein in a cell comprising contacting said cell with an effective amount of at least two anti-miRNAs, wherein said at least two anti-miRNAs target miRNAs which bind to the mRNA of said target protein, and wherein each of said at least two anti-miRNAs partially inhibits the silencing activity of said miRNAs.

In another aspect, the present invention provides a method for treating a disease or condition in a subject in need thereof, wherein said method comprises a step of partially inhibiting the silencing activity of at least two miRNAs which bind to the mRNA of a target protein associated with said disease or condition, and wherein said step of partially inhibiting the silencing activity comprises administering to said subject a therapeutically effective amount of at least two anti-miRNA which target said at least two miRNAs.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a schematic representation of a non-limiting exemplary embodiment of the invention. miR-X, miR-Y, and miR-Z, are directed to SIRT6 mRNA (the target gene) as well as to the mRNA of other, non-target genes. While SIRT6 mRNA is recognized and suppressed by all three miRs, the other, non-target mRNAs are not recognized and suppressed by all three miRs. Administration of a combination of three respective anti-miRs (e.g., Anti-miR-X, Anti-miR-Y, and Anti-miR-Z) causes an additive effect on their shared mRNA target (SIRT6 mRNA) thereby increasing SIRT6 levels (schematically represented by a bold arrow) while causing only a minor increase in each of the non-target genes (schematically represented by thin arrows).

FIG. 2 is a schematic representation showing SIRT6 mRNA level (presented as % of control) in primary mouse hepatocytes transfected with various concentrations (1, 10, 50, 75, 100, 150 nM) of anti-miR-33A.

FIG. 3 is a schematic representation showing SIRT6 mRNA level (presented as % of control) in primary mouse hepatocytes transfected with various concentrations (1, 10, 50, 75, 100, 150 nM) of anti-miR-122.

FIG. 4 is a schematic representation showing SIRT6 mRNA level (presented as % of control) in primary mouse hepatocytes transfected with various concentrations (75, 100, 150 nM) of anti-miR-33A, anti-miR-122, or a combination of anti-miR-33A and anti-miR-122 (the combination includes 75, 100, and 150 nm of each of anti-miR-33A, and anti-miR-122).

FIG. 5 is a schematic representation showing SIRT6 protein level (presented as % of control) in primary mouse hepatocytes transfected with various concentrations (75, 100 150 nM) of anti-miR-33A, anti-miR-122, or a combination of anti-miR-33A and anti-miR-122 (the combination includes 75, 100, and 150 nm of each of anti-miR-33A, and anti-miR-122).

FIGS. 6A-6E are graphs presenting expression levels of markers of pathology in mice fed with MC-Reduced HFD for six weeks. (A) Expression levels of MCP-1, TNF alpha (TNF-α), F4/80 and CD8a in MC-Reduced HFD mice (n=5) compared to normal (control) mice (n=3). Results, normalized to geometric (Geo) mean of Importin 8 (IPO8) & Ribosomal Protein Lateral Stalk Subunit P0 (RpIPO) housekeeping genes, are Average±SD fold to mRNA levels in normal mice (control) as examined by RT-qPCR. (B) Expression levels of Tissue inhibitors of metalloproteinases (TIMP-1), Collagen Type I, TR7 and TGF-b in MC-Reduced HFD mice (n=5) compared to normal (control) mice (n=3). Results, normalized to Geo mean of IPO8 & RplPO genes, are Average+SD fold to mRNA levels in normal mice (control) as examined by RT-qPCR. (C) Sirius Red staining (Arrows point at Collagen filaments) (Magnification×10). (D) Levels of serum Triglycerides (mg/dl), SGOT, and SGPT (measured in IU/L). (E) H&E staining of liver tissue sections (Magnification×10).

FIGS. 7A-7B are graphs showing reduced liver toxicity in MC-Reduced-HFD mice treated with a mix of anti-miR-33A and 122 (A) Expression levels of MCP-1 and (B) TNFa, at treatment initiation which is referred to as the background (6 weeks under diet) (n=5) compared to Control vehicle (N=8) and anti miR mix treated mice (N=8) or Negative control anti miR (N=8) at week 12 under diet. Results, one week after the last injection of the anti miR mix, normalized to Geo mean of IPO8 & RplPO genes, are Average±SE fold to control mRNA levels as examined by RT-qPCR. T test-values are presented.

FIGS. 8A-8D are graphs showing reduced fibrosis in the liver of MC-Reduced-HFD mice treated with a mix of anti-miR-33A and 122 (A) Expression levels of TIMP-1 (B) Collagen Type I (C) TR7 and (D) TGFβ markers, at treatment initiation referred to as background (6 weeks under diet) (n=5) compared to Control vehicle (N=8) and anti miR mix treated mice (N=8) or Negative control anti miR (N=8) at week 12 under diet. Results, one weeks after the last injection of the anti miR mix, normalized to Geo mean of IPO8 & RplPO genes, are Average±SE fold to control mRNA levels as examined by RT-qPCR. T test-values are presented.

FIGS. 9A-9B are graphs showing reduced (A) Liver weight (grams) and (B) Liver to Body weight ratio of MC-Reduced-HFD mice treated with mix of anti-miR-33A and 122 (N=8) compared to Control vehicle (N=5) or Negative control anti-miR (N=8) at week 12 under diet. Results, one week after the last injection of the anti-miR mix, are Average±SE fold to control. T test-values are presented.

### DETAILED DESCRIPTION OF EMBODIMENTS

The present invention is based on the surprising finding that a combination of two specific anti-miRs increased very efficiently the expression level of SIRT6.

The specific anti-miRs of the invention bind to SIRT6 miRs (i.e., to miRs that bind SIRT6 mRNA and induce mRNA degradation and/or translational repression). In other words, the specific anti-miRs of the invention inhibit the agents that cause SIRT6 mRNA degradation and/or translational repression, and as a result, SIRT6 protein is produced in greater amounts.

Several miRs are directed to SIRT6 mRNA (the target gene as defined herein). The silencing of a single miR using a corresponding anti-miR may indeed increase the expression level of the target protein (e.g., SIRT6 in the context of the present invention) but often results in a partial and insufficient increase. Even an administration of a high dose of a single anti-miR will not necessarily achieve the desired increase in the target protein levels.

Moreover, administration of a high dose of a single anti-miR may have another undesired effect. Since any specific miRNA can potentially bind many mRNAs, the miRs that are directed to SIRT6 mRNA may also bind and cause the degradation and/or translational repression of mRNA of other, non-target (also termed off-target) genes.

Therefore, administration of high concentrations of the single anti-miR may result in increased expression of non-related genes (in addition to the target gene) leading to potential undesired side-effects.

To overcome this potential detrimental effect, the present invention provides a combination of anti-miRs directed to several miRs that cause degradation and/or translational repression of SIRT6 mRNA.

Without wishing to be bound by theory, the use of a combination of several anti-miRs, each anti-miR targeting a SIRT6 miR, allows to reduce the amount of each of the anti-miRs, such that their combined additive effect causes the desired increase in SIRT6 expression. However, since the concentration of each of the anti-miRs is low, they do not affect in a similar manner the non-target genes and thus the undesired side-effects are avoided.

Moreover, by using a combination of several anti-miRs not only the effect on off-target genes is reduced but also the overall expression level of the target gene (e.g., SIRT6) is significantly increased and may reach, for example, levels which are up to 4 or 5 times higher than the regular SIRT6 expression. Such high levels cannot be obtained using a single anti-miR.

The increase in the expression level of SIRT6 can thus be carefully monitored by adjusting the relative amount of each of the anti-miRs in the mixture such that an optimal expression level is achieved with minimal toxicity and side effects.

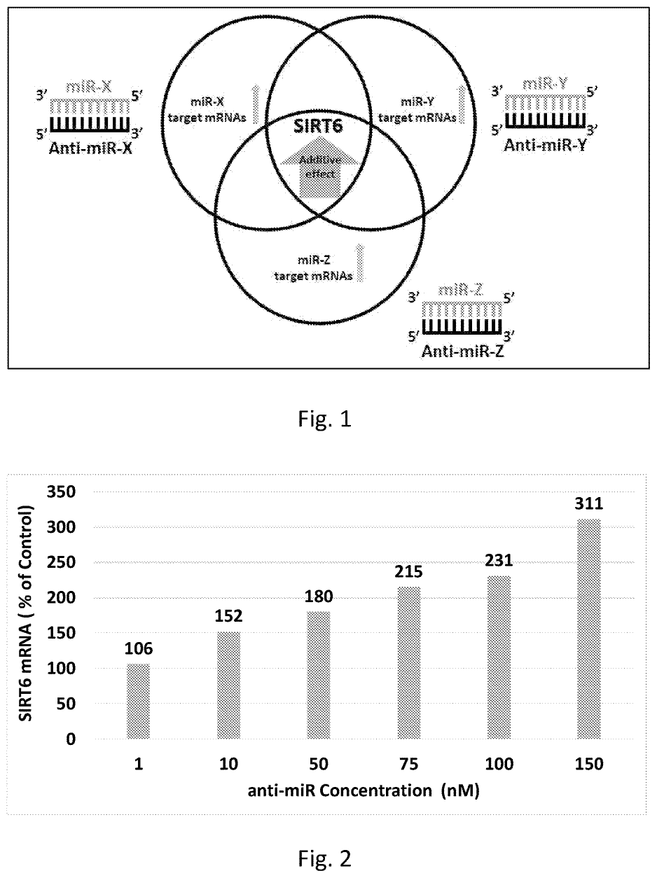

Therefore, in accordance with the invention a combination of two or more anti-miRs is used to target the SIRT6 miRs. As exemplified in FIG. 1, administration of a combination of two or more anti-miRs of the invention causes an additive effect on the shared mRNA target (namely, SIRT6 mRNA) thereby increasing SIRT6 levels while causing only a minor increase in each of the non-target genes.

Accordingly, without wishing to be bound by theory, the main advantage of combining several anti-miRs is that it causes a selective additive effect only on the target gene thereby achieving the desired increase in the SIRT6 protein level with only partial suppression of the target miRs. As a result, the off-target effect (the effect on non-target genes) is reduced.

MicroRNAs (also referred to interchangeably herein as miRNAs or miRs) are a class of short non-coding RNA molecules (17-25 nucleotides) which bind to a portion of mRNA and cause mRNA degradation and/or translational repression, thereby effectively “silencing” a gene, i.e., negatively regulate their target gene. Generally, miRNA binds to the complementary sequences of 3′-untranslated regions (3′-UTR) of their target mRNA. Many naturally occurring miRNA sequences have been identified to regulate genes in the human genome.

An anti-miR is a short synthetic RNA that is complementary to a specific miRNA target and inhibits the miRNA silencing activity.

As shown in the Examples below, the inventors have demonstrated for the first time, a robust increase in SIRT6 expression brought about by administration of a combination of anti-miRs. The desired increase of SIRT6 protein level was achieved with only partial suppression of the target miRs.

SIRT6 expression increased to a desired level that has valuable therapeutic implications. Accordingly, the anti-miR combination of the invention can be used for treating several diseases as well as to serve as anti-aging agents promoting longevity.

The ability to combine several anti miRs directed to SIRT6 miRs in accordance with the invention allows for a tailor-made combination for various diseases, and to personalize treatment according to a specific subject's needs.

Accordingly, in a first of its aspects, the present invention provides a method for treating a disease or condition in a subject in need thereof comprising a step of partially inhibiting the silencing activity of at least two SIRT6-targeted miRNAs in said subject, thereby increasing SIRT6 protein levels.

In accordance with the invention, the method comprises administering to said subject a therapeutically effective amount of at least two antisense oligonucleotides which target said at least two SIRT6-targeted miRNAs.

It is to be understood that the terms “treat”, “treating”, “treatment” or forms thereof, as used herein, mean reducing, preventing, curing, reversing, ameliorating, attenuating, alleviating, minimizing, suppressing, or halting the deleterious effects of a disease or a condition or delaying the onset of one or more clinical indications of a disease or disorder, as defined herein.

As used herein the term disease or condition generally refers to a disease or condition that can benefit from an increase in sirtuin-6 (SIRT6) upregulation.

The following are non-limiting examples of a disease or condition in accordance with the invention: cancer (e.g., colorectal cancer, lung cancer, hepatocellular carcinoma), ischemic tissue damage (e.g., intestinal ischemia-reperfusion (II/R) injury), short bowel syndrome, degenerative bone diseases (e.g., intervertebral disc degeneration (IDD)), shock), liver diseases, frailty syndrome, Alzheimer's disease, atherosclerosis, lung fibrosis, and dermal conditions (e.g. collagen depletion, UV damage). The terms “liver diseases” or “hepatic pathologies” are used interchangeably herein and relate to any disorder of the liver. Liver diseases generally include cirrhosis, or scarring of the liver, inflammation (hepatitis) due to infectious agents (e.g., hepatitis B or hepatitis C) or non-infectious causes (chemical or autoimmune hepatitis including alcoholic steatohepatitis), tumors, benign and malignant (liver cancer) and metabolic disorders. In certain embodiments, the invention provides a combination of at least two anti-miRs for use in the treatment of liver diseases such as non-alcoholic fatty liver disease (NAFLD, including nonalcoholic fatty liver (NAFL) and nonalcoholic steatohepatitis (NASH)), cholestasis and intrahepatic cholestatic liver diseases such as primary sclerosing cholangitis (PSC) and primary biliary cirrhosis (PBC).

Nonalcoholic steatohepatitis, or NASH, is the most severe form of nonalcoholic fatty liver disease (NAFLD), a condition in which the liver builds up excessive fat deposits. Excessive fatty liver accompanied by inflammation, is defined as NASH.

As shown in Example 2 below, administration of a combination (namely, a mixture) of anti-miR-33A and anti-miR-122 was highly effective in treating/reducing symptoms of NASH in an animal model. Accordingly, in a specific embodiment the concerns methods and uses of a combination of at least two anti-miRs (e.g., anti-miR-33A and anti-miR-122) in treating NASH and/or reducing symptoms associated with NASH, such symptoms include but are not limited to:
a. reducing liver inflammation, as manifested for example by reduction in immune/inflammatory cell infiltration into the liver and/or secretion of inflammatory cytokines, such as TNFa or MCP-1 by liver cells.
b. reducing TGF-β levels in the liver.
c. reducing fibrosis as manifested for example by reducing TIMP-1, Collagen Type I and TR7 gene expression in the liver.
d. reducing fat accumulation in the liver, as evidenced for example by reduced liver weight, and normalized ratio between liver and body weight.

As used herein the term “silencing activity” refers to the ability of miRNAs to inhibit the expression of a target gene by causing mRNA degradation and/or translational repression.

As described above, antisense oligonucleotides (e.g., anti-miRNAs) may interact with miRNAs and thereby inhibit their silencing activity.

Such inhibition may be a “complete inhibition” whereby substantially all the specific miRNAs that are recognized by a certain antisense oligonucleotide (e.g., a specific anti-miRNAs) are inhibited and as a result the expression levels of all the target genes that are affected by said specific miRNA are significantly increased. Such an effect can be achieved for example by administering a large amount of the specific antisense oligonucleotide, for example such that the amount of the antisense oligonucleotide exceeds that of the miRNA to ensure complete inhibition.

The inhibition of silencing activity may also be a partial inhibition. Accordingly, as used herein the term “partially inhibiting the silencing activity” refers to an incomplete inhibition whereby not all the specific miRNAs that are recognized by a certain antisense oligonucleotide are inhibited. As a result, the expression level of the genes that are affected by said specific miRNA is differentially affected whereby the expression level of some of these genes will be increased while others will remain unaffected or affected at a lower degree. Such an effect can be achieved for example by administering a small amount of the specific antisense oligonucleotide, for example such that the amount of the antisense oligonucleotide will not exceed that of the miRNA, or will neutralize only a fraction of the miRNA, to ensure partial inhibition.

In such case, several different antisense oligonucleotides (e.g., anti-miRNAs) can be administered such that their effect on the target gene is concerted to achieve the desired outcome, e.g., an increase in the level of expression of the target gene (e.g., SIRT6) while having a reduced effect or no effect on the level of expression of other genes.

The exact amounts of each of the antisense oligonucleotides (e.g., anti-miRNAs) can be determined based on the disease or disorder to be treated, and/or be personalized to a specific subject, and/or be determined based on in vitro and/or in vivo experiments, for example the experiments shown in the Examples below. For example, to determine the suitable amount of anti-miR which is capable of partially inhibiting the silencing activity of the corresponding miR, an in vitro calibration test can be designed in which different amounts of the anti-miR are tested for their ability to inhibit miR activity. An amount that causes total suppression is defined as 100% and accordingly an amount that causes a partial suppression (e.g., 10%, 20%, 30%, 40%, 50%, 60%, 70%, 80%, 90% etc.) can be determined.

Therefore, a “therapeutically effective amount” of the isolated anti miRNA according to the invention, or the pharmaceutical composition according to the invention for purposes herein defined is determined by such considerations as are known in the art in order to cure, arrest or at least alleviate or ameliorate the medical condition. For any preparation used in the methods of the invention, the dosage or the therapeutically effective amount can be estimated initially from in vitro cell culture assays or based on suitable animal models.

In some embodiments the isolated anti miRNA according to the invention or pharmaceutical composition according to the invention is administered to the subject as a single dose or in multiple doses.

The terms “SIRT6 miRNAs” and “SIRT6-targeted miRNAs” are used interchangeably herein and refer to any miRNA that can potentially affect SIRT6 mRNA degradation and/or cause SIRT6 mRNA translational repression. The following is a non-limiting list of SIRT6 miRs:

miR-33A
(SEQ ID NO: 1)
(GUGCAUUGUAGUUGCAUUGCA),
miR-33B
(SEQ ID NO: 2)
(GUGCAUUGCUGUUGCAUUGC),
miR-122
(SEQ ID NO: 3)
(UGGAGUGUGACAAUGGUGUUUG),
miR-370
(SEQ ID NO: 4)
(GCCUGCUGGGGUGGAACCUGGU),
miR-34c-5p
(SEQ ID NO: 28)
(AGGCAGUGUAGUUAGCUGAUUGC),
miR-351-5p
(SEQ ID NO: 29)
(UCCCUGAGGAGCCCUUUGAGCCUGA),
miR-378b
(SEQ ID NO: 30)
(ACUGGACUUGGAGGCAGAA),
miR-186-5p
(SEQ ID NO: 31)
(CAAAGAAUUCUCCUUUUGGGCU),
miR-34a-5p
(SEQ ID NO: 32)
(UGGCAGUGUCUUAGCUGGUUGU),
miR-125b-5p
(SEQ ID NO: 33)
(UCCCUGAGACCCUAACUUGUGA),
miR-495-3p
(SEQ ID NO: 34)
(AAACAAACAUGGUGCACUUCUU),
miR-766-3p
(SEQ ID NO: 35)
(ACUCCAGCCCCACAGCCUCAGC),
miR-25-3p
(SEQ ID NO: 36)
(CAUUGCACUUGUCUCGGUCUGA),
miR-338-3p
(SEQ ID NO: 37)
(UCCAGCAUCAGUGAUUUUGUUG),
miR-92a-3p
(SEQ ID NO: 38)
(UAUUGCACUUGUCCCGGCCUGU),
miR-10396b-5p
(SEQ ID NO: 39)
(CGGCGGGGCUCGGAGCCGGG),
miR-6787-5p
(SEQ ID NO: 40)
(UGGCGGGGGUAGAGCUGGCUGC),
miR-1908-5p
(SEQ ID NO: 41)
(CGGCGGGGACGGCGAUUGGUC),
miR-663a
(SEQ ID NO: 42)
(AGGCGGGGCGCCGCGGGACCGC),
miR-541-3p
(SEQ ID NO: 43)
(UGGUGGGCACAGAAUCUGGACU),
miR-654-5p
(SEQ ID NO: 44)
(UGGUGGGCCGCAGAACAUGUGC).

Additional SIRT6 miRs can be identified using bioinformatic methods (non-limiting examples include miRNA target prediction database (MiRDB), and TargetScan).

As used herein the term “increase” or “increasing” refers to an augmentation in the expression level of SIRT6 mRNA or the translation level of SIRT6 protein, or generally to SIRT6 protein level.

By the term “increase” or “increasing” in the context of the present invention it is meant that the isolated anti-miRNAs of the invention augments or boosts by at least about 25%, 50%, 75%, 100%, 125%, 150%, 175%, 200%, 225%, 250%, 275%, 300%, 325%, 350%, 375%, 400%, 425%, 450%, 475%, 500% or more, the expression level of SIRT6 mRNA or the translation level of SIRT6 protein or generally the SIRT6 protein level, as compared to the expression level of SIRT6 mRNA or translation level of SIRT6 protein, or generally SIRT6 protein level in the absence of the antisense oligonucleotides (e.g., anti-miRNAs) of the invention.

In specific embodiments the at least two antisense oligonucleotides (e.g., anti-miRNAs) of the invention cause an increase in the expression level of SIRT6 mRNA or an increase in translation level of SIRT6 protein, or generally in SIRT6 protein level of about 100%, 200%, 300% or more.

As used herein, the term “antisense oligonucleotides” refers to a nucleic acid molecule comprising a sequence that complements the miRNA nucleic acid sequence.

In a specific embodiment, said antisense oligonucleotide is an anti-miRNA molecule. Methods for preparing anti-miRNA molecules are well known in the art, for example the anti-miRs can be synthesized by the general Solid phase technique using phosphoramidates modified oligos backbones. Oligos can be HPLC purified and evaluated by Mass spectrometry.

The following is a non-limiting list of SIRT6 anti-miRs:

Anti-miR-33A:
(SEQ ID NO: 5)
TGCAATGCAACTACAATGCAC
Anti-miR-122:
(SEQ ID NO: 6)
ACAAACACCATTGUCACACUCCA
Anti-miR-33B:
(SEQ ID NO: 45)
GCAATGCAACAGCAATGCAC,
Anti-miR-370:
(SEQ ID NO: 46)
ACCAGGTTCCACCCCAGCAGGC,
Anti-miR-34c-5p:
(SEQ ID NO: 47)
GCAATCAGCTAACTACACTGCCT,
Anti-miR-351-5p:
(SEQ ID NO: 48)
TCAGGCTCAAAGGGCTCCTCAGGGA,
Anti-miR-378b:
(SEQ ID NO: 49)
TTCTGCCTCCAAGTCCAGT,
Anti-miR-186-5p:
(SEQ ID NO: 50)
AGCCCAAAAGGAGAATTCTTTG,
Anti-miR-34a-5p:
(SEQ ID NO: 51)
ACAACCAGCTAAGACACTGCCA,
Anti-miR-125b-5p:
(SEQ ID NO: 52)
TCACAAGTTAGGGTCTCAGGGA,
Anti-miR-495-3p:
(SEQ ID NO: 53)
AAGAAGTGCACCATGTTTGTTT,
Anti-miR-766-3p:
(SEQ ID NO: 54)
GCTGAGGCTGTGGGGCTGGAGT,
Anti-miR-25-3p:
(SEQ ID NO: 55)
TCAGACCGAGACAAGTGCAATG,
Anti-miR-338-3p:
(SEQ ID NO: 56)
CAACAAAATCACTGATGCTGGA,
Anti-miR-92a-3p:
(SEQ ID NO: 57)
ACAGGCCGGGACAAGTGCAATA,
Anti-miR-10396b-5p:
(SEQ ID NO: 58)
CCCGGCTCCGAGCCCCGCCG,
Anti-miR-6787-5p:
(SEQ ID NO: 59)
GCAGCCAGCTCTACCCCCGCCA,
Anti-miR-1908-5p:
(SEQ ID NO: 60)
GACCAATCGCCGTCCCCGCCG,
Anti-miR-663a:
(SEQ ID NO: 61)
GCGGTCCCGCGGCGCCCCGCCT,
Anti-miR-541-3p:
(SEQ ID NO: 62)
AGTCCAGATTCTGTGCCCACCA,
Anti-miR-654-5p:
(SEQ ID NO: 63)
GCACATGTTCTGCGGCCCACCA.

In all sequences T (Thymidine) can be replaced with U (Uridine)

In an embodiment, the anti-miRs of the invention comprise the sequence denoted as SEQ ID NO: 5 or SEQ ID NO: 6. In another embodiment, the anti-miRs of the invention consist of the sequence denoted as SEQ ID NO: 5 or SEQ ID NO: 6.

The present invention also encompasses anti-miR molecules having at least 75%, at least 80%, at least 85%, or at least 90% sequence homology to the anti-miR molecules of the invention. In an embodiment, the present invention refers to anti-miR molecules having at least 75%, at least 80%, at least 85%, or at least 90% sequence homology to any one of SEQ ID Nos 5 or 6.

The present invention also encompasses variants of the anti-miR molecules. The variants may include nucleic acid substitutions which do not alter the activity of the anti-miRs herein described, e.g., miR inhibition.

The term “variant” refers to an anti-miR molecule in which one or more nucleotides are deleted, substituted, or added, wherein these alterations do not abolish the activity of the anti-miRs herein described.

It should be appreciated that by the term “added”, as used herein it is meant any addition of one or more nucleic acids to the sequences described herein.

It should be appreciated that by the term “substituted”, as used herein it is meant any substitution of one or more nucleic acids of the sequences described herein.

It should be appreciated that the term “deleted”, as used herein, refers to any deletion of one or more nucleic acids from the sequences described herein.

The present invention also encompasses an active fragment of any of the anti-miRs of the invention. The term “active fragment” refers to a portion of the anti-miR that maintains the activity of the complete anti-miR, e.g., miR inhibition.

The present invention also encompasses various modifications of the anti-miR which may increase the molecule's stability. New classes of chemically modified anti-miRNA oligonucleotides are described for example in Lima et al (2018, RNA Biol. 15 (3) 338-352). Non-limiting examples of chemical modifications include sugar modifications, e.g., 2′ O-methyl oligoribonucleotides (O-Me), 2′ O-methoxyethyl-RNA (MOE), 2′-F modifications, locked nucleic acids (LNA), and combinations thereof, and backbone modifications e.g., phosphorothioate (PS) linkage, Morpholino oligonucleotides, and phosphonoacetate oligonucleotides (PACE).

Administration according to the present invention may be performed by any of the following routes: oral administration, intravenous administration, intramuscular administration, intraperitoneal administration, intrathecal administration, subcutaneous administration, intra-rectal administration, intranasal administration (e.g., by inhalation using an aspirator), ocular administration, or topical administration.

The anti-miRNAs as herein defined, any pharmaceutical compositions comprising the same or any conjugates comprising them may be administered to a subject prior to or post disease onset, in a single dose or in multiple doses.

In an embodiment, the anti-miR is conjugated to a targeting moiety, e.g., asialoglycoprotein. The asialoglycoproteins are natural binding targets to the liver Asialoglycoprotein receptor 1 and 2 (ASGPR1-2) (Aaron D. Springer and Steven F. Dowdy, Nucleic Acid Therapeutics, Vol. 28, No. 3, June 2018). After binding to the ASGPR the oligonucleotide cargo escapes to the cytoplasm to facilitate miR knockdown.

In an embodiment, the anti-miR is conjugated to N-acetylgalactosamine (GalNAc) that binds to the asialoglycoprotein receptor. GalNAc may facilitate targeted delivery of the anti-miR to liver hepatocytes (Debacker et al. (2020) Molecular Therapy, Volume 28, Issue 8, pages 1759-1771).

In an embodiment, the anti-miR is conjugated to a cholesterol moiety.

In an embodiment, the anti-miR is conjugated to a Vitamin E moiety.

In some embodiments the methods according to the invention are wherein said methods further comprise administering to a subject in need thereof an additional therapeutic agent. In specific embodiments the additional therapeutic agent may be a small molecule which increases SIRT6 enzymatic activity, and in the case of cancer therapy said additional therapeutic agent may also be a chemotherapeutic agent or an immune check point modulator.

In some embodiments, the method of the invention further comprises measuring the levels of SIRT6 mRNA or the levels of SIRT6 protein in said subject, prior to and/or during treatment, wherein the types and amounts of said antisense oligonucleotides (e.g., miRNAs) are adjusted according to the measured levels of SIRT6. The levels of SIRT6 mRNA or SIRT6 protein may be measured using any method known in the art, for example using the methods demonstrated in the Examples below.

In some embodiments, the present invention provides a pharmaceutical composition comprising a combination of at least two antisense oligonucleotides which target at least two SIRT6 miRNAs and a suitable carrier or excipient or diluent for use in a method of treatment of a disease, wherein said antisense oligonucleotides cause partial inhibition of the silencing activity of said at least two SIRT6 miRNAs.

The “pharmaceutical composition” of the invention generally comprises at least two SIRT6 anti-miRNA as herein defined and a buffering agent, an agent which adjusts the osmolarity of the composition and optionally, one or more pharmaceutically acceptable carriers, excipients and/or diluents as known in the art.

As used herein the term “pharmaceutically acceptable carrier, excipient or diluent” includes any solvents, dispersion media, coatings, antibacterial and antifungal agents, and the like, as known in the art. The carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated.

In some embodiments, the anti-miR of the invention may be incorporated into lipid nanoparticles.

In other embodiments the pharmaceutical composition according to the invention further comprises an additional therapeutic agent.

### EXAMPLES

### Materials and Methods

### Isolation of Primary Murine Hepatocytes

Primary mouse hepatocytes were isolated using a method adopted from Meital Charni-Natan and Ido Glodstein (Star Protocols, Vol. 1, Issue 2, 18 Sep. 2020).

Briefly, 5-12 weeks old C57BL/6JOlaHsd mice were euthanized with 100% isofluorane. The liver was perfused with HBSS X1 (Gibco) supplemented with 2.1 g/L NaHCO₃(Sigma-Aldrich Merck), and 0.3 g/L EDTA (Sigma-Aldrich Merck) via the inferior vena cava. Pump speed was set to 4 ml/minute and the portal vein was severed to allow drainage. 50 ml of HBSS were allowed to flow and then another 50 ml of Liver digest medium (Gibco) were allowed to perfuse.

The liver was extracted and manually disrupted with tweezers in 12 ml of liver growth media (LGM-DMEM supplemented with 10% FBS, 2% pen/strep, 1 mM pyruvate, 1 μM dexamethasone and 100 nM Insulin). The cell suspension was passed on a 70 μm filter and live Hepatocytes were isolated by Percoll gradient procedure. Cells were washed and plated on Collagen coated sterile 12 well plates. 200,000 cells were seeded in each well and grown in LGM.

Transfection of Primary Hepatocytes with Anti miR

The anti-Mirs were synthesized by the general Solid phase technique using phosphoramidates modified oligos backbones. Oligos were HPLC purified and evaluated by Mass spectrometry. 24 hours after seeding, the cells were transfected with anti miR-33A (SEQ ID NO: 5), anti miR-122 (SEQ ID NO: 6) or a combination of anti miR-33A and anti miR-122 (at final concentrations of 1, 10, 50, 75, 100 and 150 nM). Anti miR-33A and anti miR-122 are complementary to miR-33A (Horizon), and miR-122 (manufactured by Axolabs Gmbh), respectively. The transfection was carried out using Lipofectamine RNAiMAX Transfection Reagent (Thermo fisher scientific) according to the manufacturer protocol with minor changes. The total Anti-miR transfected at each well is indicated.

Briefly, 2.8 μl of RNAimax was diluted in Opti-Mem medium. The Anti-miRs were also diluted in Opti-Mem medium. Diluted RNAimax was added to each diluted anti-miR, and complexes were allowed to form for 15 minutes. A total of 150 μl diluted complexes were added to each well. Total transfection volume was 1.150 ml.

The cells were harvested 48 hours post transfection for further analysis in Western blot or qRTPCR.

### RNA Extraction

48 hours after transfection, RNA was isolated from the cells using the PureLink™ RNA Isolation Kit (Thermo fisher scientific cat-12183018A) according to the manufacturer protocol.

### Quantitative Real-Time PCR

cDNA was prepared and diluted 1:20 with DDW free of nucleases (5 μl cDNA to 95 μl H2O). The following primers were prepared and diluted to a concentration of 20 μM:

SIRT6 Forward primer:
SEQ ID NO: 7
CAGAGCTGCACGGAAACATG
SIRT6 Reverse primer:
SEQ ID NO: 8
TCATCAGCGAGCATCAGGTC,
beta actin Forward primer:
SEQ ID NO: 9
AGCCATGTACGTAGCCATCC,
beta actin Reverse primer:
SEQ ID NO: 10
CTCTCAGCTGTGGTGGTGAA,
MCP-1 Forward primer:
SEQ ID NO: 12
AGGTCCCTGTCATGCTTCTG,
MCP-1 Reverse primer:
SEQ ID NO: 13
TCTGGACCCATTCCTTCTTG,
CD8a Forward primer:
SEQ ID NO: 14
ACTACCAAGCCAGTGCTGCGAA,
CD8a Reverse primer:
SEQ ID NO: 15
ATCACAGGCGAAGTCCAATCCG,
TNF-a Forward primer:
SEQ ID NO: 16
CGTCAGCCGATTTGCTATCT,
TNF-a Reverse primer:
SEQ ID NO: 17
CGGACTCCGCAAAGTCTAAG,
TIMP-1 Forward primer:
SEQ ID NO: 18
ATTCAAGGCTGTGGGAAATG,
TIMP-1 Reverse primer:
SEQ ID NO: 19
CTCAGAGTACGCCAGGGAAC
TR7 Forward primer:
SEQ ID NO: 20
CACCTTCCTCTGGCACAGTTAC,
TR7 Reverse primer:
SEQ ID NO: 21
TCCCTTCCTCGATGCCACTT
TGFβ Forward primer:
SEQ ID NO: 22
TTGCTTCAGCTCCACAGAGA,
TGFβ Reverse primer:
SEQ ID NO: 23
TGGTTGTAGAGGGCAAGGAC
IPO8 Forward primer:
SEQ ID NO: 24
CGTGACAGTAGATACCAACGCTC,
IPO8 Reverse primer:
SEQ ID NO: 25
CATAGCACTCGGCATCTTCTCC
RplPO Forward primer:
SEQ ID NO: 26
GCTTCGTGTTCACCAAGGAGGA,
RplPO Reverse primer:
SEQ ID NO: 27
GTCCTAGACCAGTGTTCTGAGC

15 μl of reaction MIX (PowerUp™ SYBR™ Green Master Mix, ThermoFisher) containing 1.5 μl forward primer and 1.5 μl reverse primer were placed in each well in a PCR plate (96 well plate). 5 μl of diluted cDNA were added to each well to a final volume of 20 μL. The plate was centrifuged for 2 min at 1200 RPMI (at RT). The PCR reaction was performed in a CFX Connect Real-Time PCR Detection System (Bio-Rad) according to the manufacturer's protocol. Real time PCR amplicons were visualized by Sybr green fluorescence and quantified with the Bio-rad CFX maestro program.

The program was run with the following step parameters:
1. 50° C. for 2 min
2. 95° C. for 2 min
3. 95° C. for 15 sec
4. 60° C. for 1 min

Steps 3 and 4 were repeated 40 times.

### Western Blot Analysis

48 hours after transfection, Western blot was performed using the following procedure: The cells' supernatant was removed and the primary hepatocytes were lysed by adding lysis buffer (50 mM Tris-HCl, pH 7.4, 150 mM NaCl, 1 mM DTT, 10% Glycerol, 1 mM MgCl2, 0.1% NP-40) to the wells. The cells were scraped off the plate, transferred to a microcentrifuge tube, and incubated on ice for 10 min. The tubes were sonicated for 30 sec. to complete cell lysis and shear DNA (to reduce sample viscosity). Lysates were cleared by centrifugation (17,000G, at 4° C. for 10 minutes). For direct electrophoretic analysis, Laemmli sample buffer was added to cell lysates, that were subsequently resolved by SDS acrylamide gel and transferred to a nitrocellulose membrane by electrophoresis using the Trans-Blot Turbo System (Bio-Rad). Membranes were blocked in 5% non-fat (skim) milk (Difco) or 5% bovine serum albumin (BSA) (MPB) in 1× Tris buffered saline with Tween 20 (TBST) for 1 hour at room temperature and incubated with a primary anti SIRT6 rabbit monoclonal antibody (cat-12486 Cell Signaling) or Tubulin rabbit monoclonal antibody (cat-2128 Cell Signaling) overnight in blocking buffer (TBST with 5% w/v with BSA) at 4° C., washed in TBST, and incubated for 1 hour with a secondary anti mouse antibody linked to horseradish peroxidase. Immunoreactive bands were detected using enhanced chemiluminescence (ECL) reagents (Bio-Rad).

### Example 1: Induction of Increased Expression of SIRT6 by a Combination of Anti miRs

Primary mouse hepatocytes were produced as described above and were transfected with the following oligonucleotides: anti-miR-33A, anti-miR-122, a combination of anti-miR-33A and anti-miR-122, or a negative control sequence. The negative control sequence is a non-targeting sequence with a low RNA homology pattern: ACCAUAUUGCGCGUAUAGUCGC (SEQ ID NO: 11). For examining the effect on SIRT6 mRNA, 48 hours after transfection, RNA was extracted from the hepatocytes, quantified, and reverse transcribed. Subsequently, SIRT6 mRNA levels were quantified by a Sybr green real-time PCR assay.

For examining the effect on SIRT6 protein, 48 hours after transfection hepatocytes were lysed in lysis buffer, and the total protein content was measured. Then, SIRT6 protein levels were quantified by SDS-PAGE.

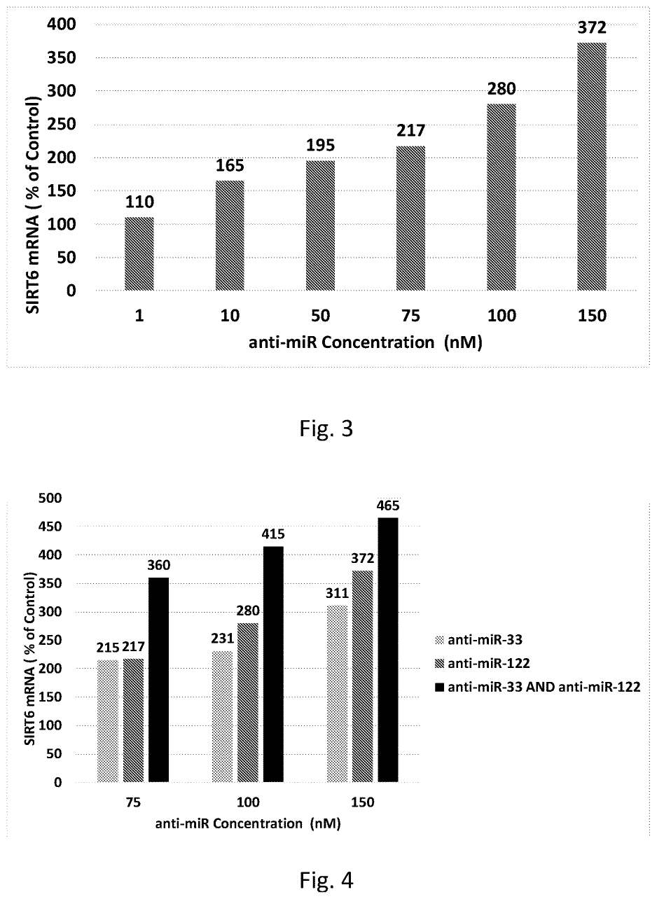

SIRT6 mRNA level increased in a dose-dependent manner after transfection with anti-miR-33A (FIG. 2) or anti-miR-122 (FIG. 3) in the primary mouse hepatocytes. The cells were transfected with increasing concentrations of the oligonucleotides (1, 10, 50, 75, 100 and 150 nM).

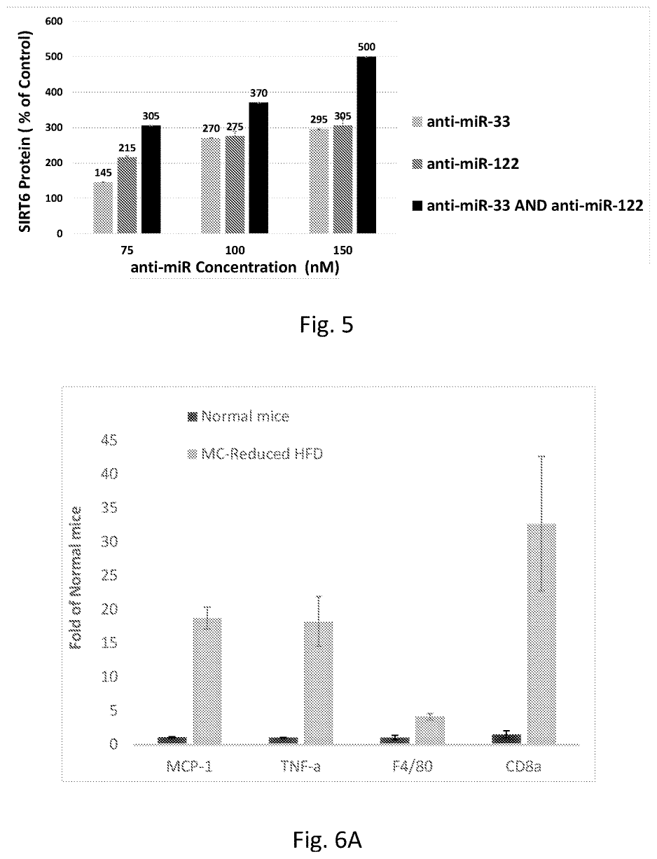

Of note, co-transfection with anti-miR-33A and anti-miR-122 (75, 100 and 150 nM each) lead to a potent upregulation of SIRT6 mRNA levels (FIG. 4) and protein levels (FIG. 5) in the primary mouse hepatocytes.

### Example 2: Inducing SIRT6 Expression in the Liver in the MC-Reduced HFD Mouse Model of NASH Improves Multiple Markers of NASH Disease

The effects of inducing SIRT6 expression on liver inflammation and fibrosis formation were assessed in a mouse model of NASH.

In this model, mice are fed with choline-deficient (CD) reduced Methionine (0.1%) diet (MC-Reduced HFD), which produces a progressive liver pathology characterized by the development of steatosis with inflammation and fibrosis within a short time frame [14].

Accordingly, C57BL/6J Male mice at week 10 of age, were fed with L-Amino Acid Diet With 45 kcal % Fat With 0.1% Methionine and No Added Choline (Research Diet, Cat #A06071309i).

Anti-miR-33A (SEQ ID NO: 5) and anti-miR-122 (SEQ ID NO: 6), as well as a negative control sequence (SEQ ID NO: 11) were fully phosphothioated and modified as follows:

Anti miR-33A (SEQ ID NO: 5):
TmGmCfAfAfTfGfCfAfAfCmTmAmCfAfAfTfGfCfAmCm (also referred to herein as modified SEQ ID NO: 5)
Anti miR-122 (SEQ ID NO: 6):
AmCmAfAfAfCfAfCfCfAfTmTmGmUfCfAfCfAfCfUfCfCmAm (also referred to herein as modified SEQ ID NO: 6)
Negative control sequence (SEQ ID NO: 11):

AmCmCfAfUfAfUfUfGfCfGmCmGmUfAfUfAfGfUfCfGmCm.

m-indicates a 2′ MOE modified base and f-indicates a 2′F modified base.

The modified anti miR-33A, anti miR122, and the control sequence were conjugated with GalNac (L96, Axolabs GmbH, Germany), 10 mg from each resuspended in 5 mL DDW to have 2 mg/mL stock.

The GalNac-conjugated modified anti miR-33A and anti miR122 were mixed and introduced together to C57Bl/6 mice after 6 weeks under the MC-Reduced HFD. The GalNac-conjugated mix of the two anti miR was injected at a dose of 3 mg/Kg each and the control non relevant sequence (SEQ ID NO: 11) at a dose of 6 mg/kg. The following dosing regimen was used: Three injections with 3 days separating between the injection, this regimen was repeated 14 days afterwards (namely three additional injections separated by three days between them) and two additional injections after additional 14 days (Total 8 injections) (N=8). The control sequence (SEQ ID NO: 11) was injected only as described for the first 6 injections (N=8). Mice injected with the vehicle PBS served as control (N=8).

The Mix of the two anti-miRNA was introduced to mice when NASH pathology could already be observed. Blood was collected, liver was extracted from the mice and levels of the liver enzymes alanine aminotransferase/aspartate aminotransferase (ALT/AST), fat accumulation in the liver and Sirius red staining analyses were performed (N=5).

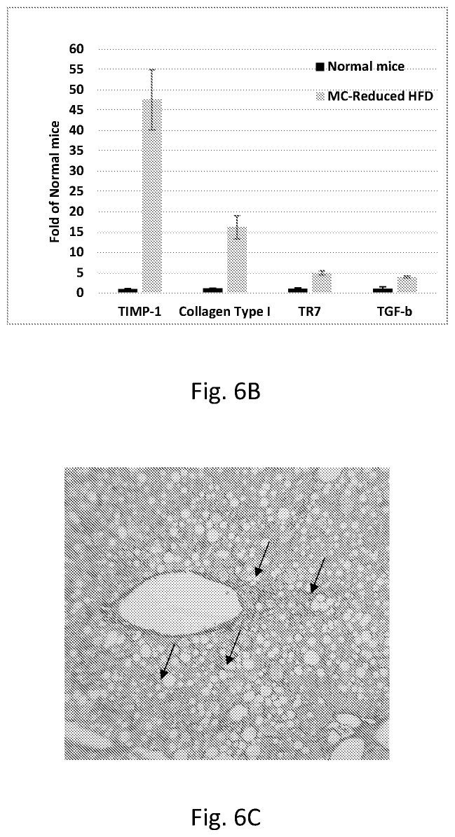

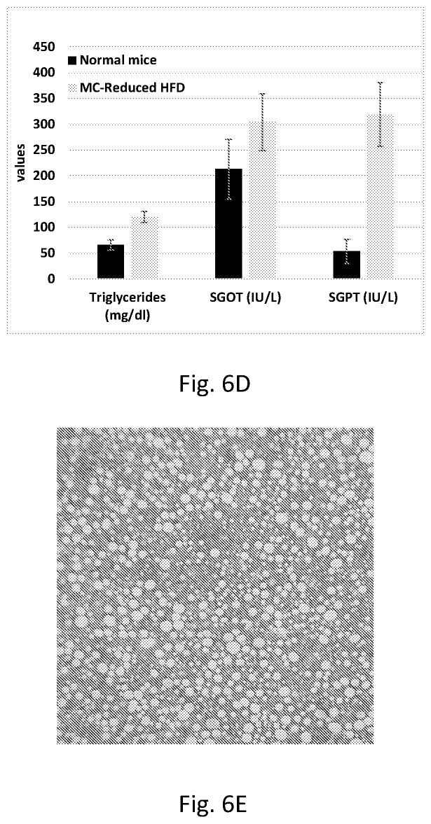

Liver inflammation was manifested by the elevation of inflammatory genes (MCP-1 and TNF-α) and the presence of infiltrating lymphocytes and macrophages indicated by the elevated levels of CD8 and F4/80 markers, respectively (FIG. 6A). Formation of fibrotic tissue was manifested by the elevation of genes involves in fibrosis (TIMP-1, Collagen Type I, TR7 and TGFβ) (FIG. 6B) and by the presence of collagen fibers after Sirius red staining (FIG. 6C). The expression levels of the genes were normalized to the Geo mean of IPO8 & RplPO genes and were examined by RT-qPCR. Liver toxicity was seen by elevated levels of liver enzymes, AST (measured using a glutamic-oxaloacetic transaminase (SGOT) test), ALT (measured using the SGPT test), in the serum (FIG. 6D). accumulation of fat was seen by measuring the triglycerides level in the serum (FIG. 6D) and the presence of hepatocytes swelling in the liver as seen in liver section after H&E staining (FIG. 6E).

One week after the last injection of the anti-miRNA mix, at week 12 under MC-Reduced HFD, mice were sacrificed and the effects on NASH pathology were investigated.

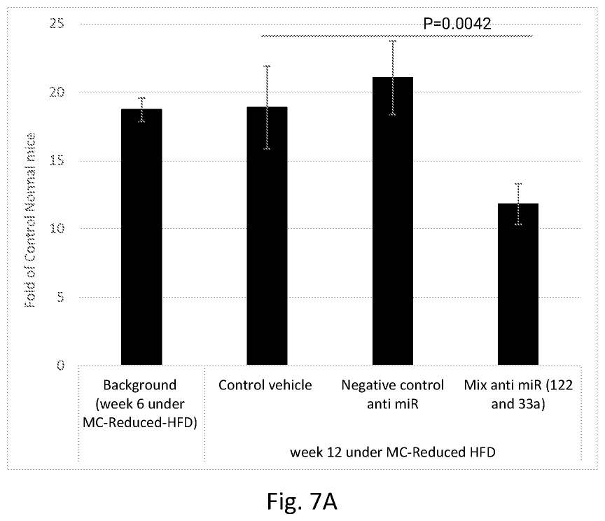

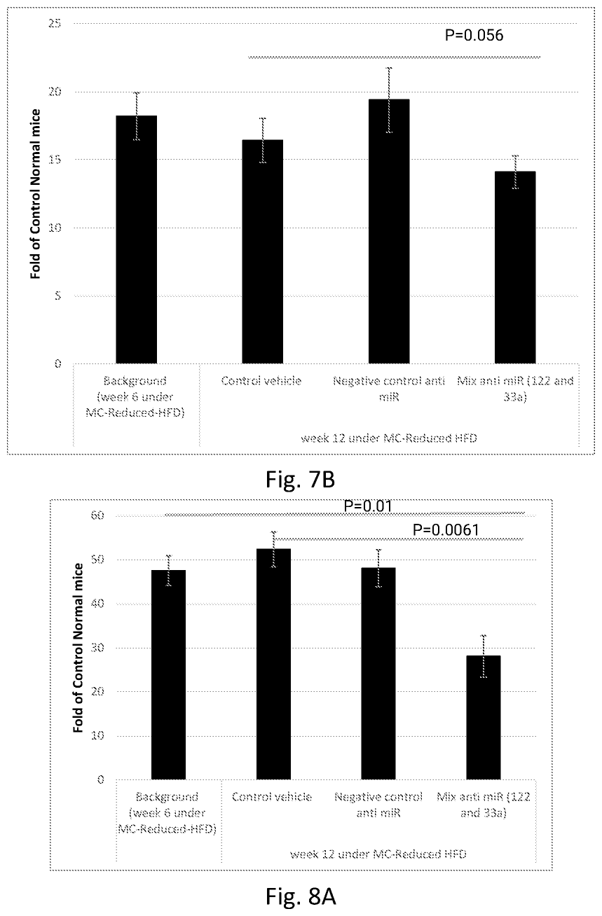

The immune reaction is represented by immune/inflammatory cell infiltrations and secretion of inflammatory cytokines by liver cells. MCP-1 and TNF-α genes are up-regulated during the inflammatory phase of NASH. These genes are induced in mice livers under MC-Reduced HFD and reduced in mice treated with the Mix of the anti-miRNA (FIGS. 7A and 7B, respectively).

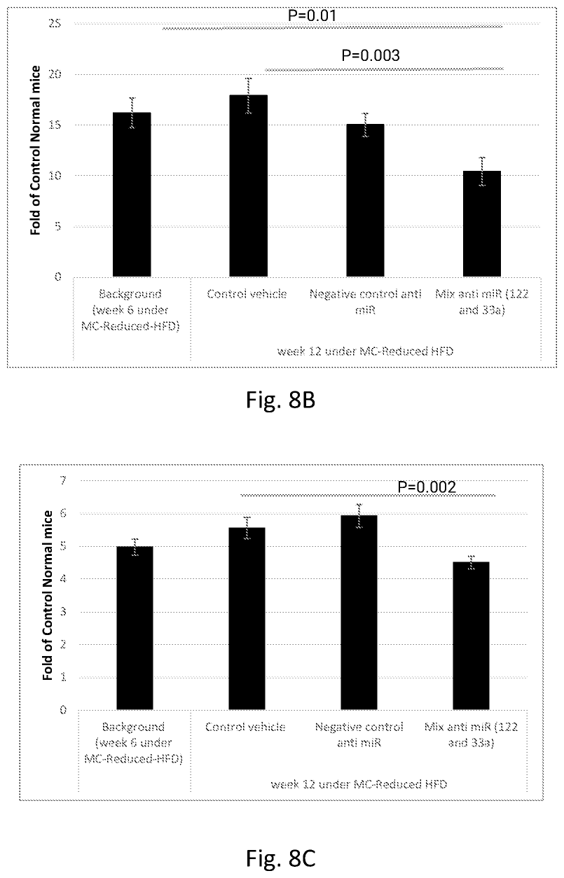

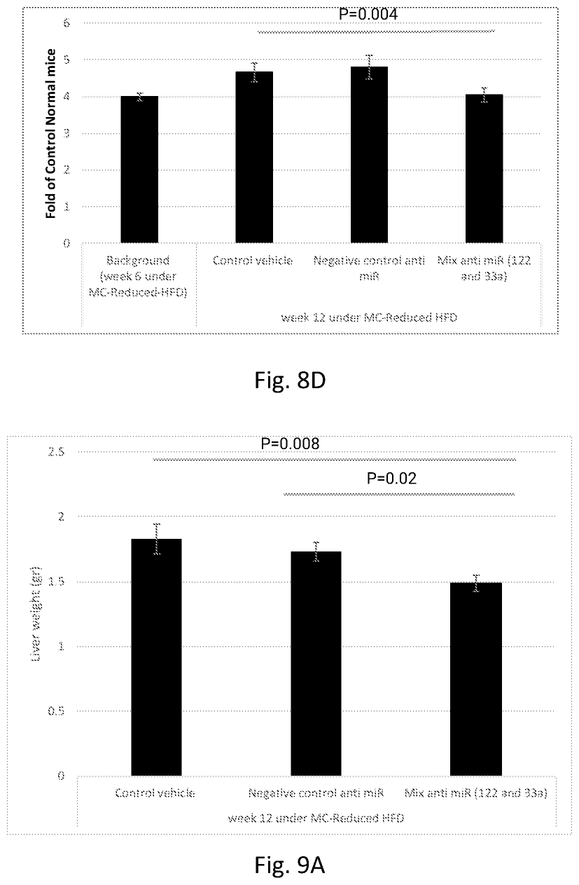

TGF-β is a central regulator in chronic liver disease contributing to all stages of disease progression from initial liver injury through inflammation and fibrosis to cirrhosis and hepatocellular carcinoma. Liver damage-induced levels of active TGF-β enhance hepatocyte destruction and mediate hepatic stellate cell and fibroblast activation. TGF-β levels in the liver were induced under MC-Reduced HFD (FIG. 6B) and reduced after treatment with the mix of the anti miR (FIG. 8D). Formation of fibrotic tissue in the liver under MC-Reduced HFD was also evaluated by measuring TIMP-1, Collagen Type I and TR7 gene expression. As seen in FIG. 6B, all genes were induced under this diet as compared with Control normal mice. Introduction of the mix anti miRNA reduced fibrosis as manifested by the significant reduction of the expression of these genes compared to their levels in the liver at the beginning of the treatment (week 6) or by the end of the study (FIG. 8A-C). The expression levels of all genes were examined using RT-qPCR.

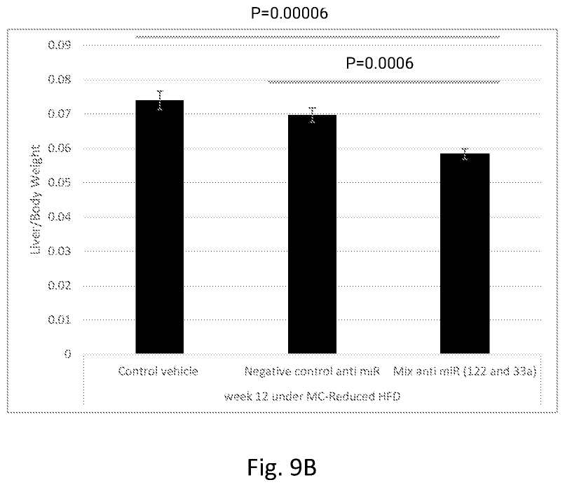

Treatment with anti miR mix reduced fat accumulation in the liver, as could be seen by reduced liver weight, and the ratio between liver and body weight compared with the non-treated controls and mice treated with the control miR sequence (FIGS. 9A and B, respectively).

1.-63. (canceled)

64. A method for treating a disease or condition in a subject in need thereof comprising a step of partially inhibiting the silencing activity of at least two SIRT6-targeted miRNAs in said subject, thereby increasing SIRT6 protein levels.

65. The method of claim 64 wherein said method comprises administering to said subject a therapeutically effective amount of at least two antisense oligonucleotides which target said at least two SIRT6-targeted miRNAs.

66. The method of claim 65 wherein said therapeutically effective amount is an amount sufficient to increase SIRT6 mRNA level or SIRT6 protein level while affecting to a lesser degree the expression of other genes.

67. The method of claim 65 wherein said method further comprises measuring the levels of SIRT6 mRNA expression or the levels of SIRT6 protein in said subject, prior to and/or during treatment, wherein the types and amounts of said antisense oligonucleotides are adjusted according to the measured levels of SIRT6.

68. The method of claim 64 wherein said disease or condition is selected from a group consisting of cancer (e.g., colorectal cancer, lung cancer, hepatocellular carcinoma), ischemic tissue damage (e.g., intestinal ischemia-reperfusion (II/R) injury), short bowel syndrome, degenerative bone diseases (e.g., intervertebral disc degeneration (IDD)), hyperglycemia (e.g., statin-induced hyperglycemia), obesity, type-2 diabetes, autoimmune and inflammatory diseases (e.g., Crohn's disease, Colitis, Pancreatitis, Septic shock), liver diseases (e.g., non-alcoholic fatty liver disease (NAFLD), NASH, fibrosis, hepatitis), frailty syndrome, Alzheimer's disease, atherosclerosis, lung fibrosis, and dermal conditions (e.g. collagen depletion, UV damage).

69. The method of claim 64 wherein said at least two SIRT6-targeted miRNA are selected from the group consisting of miR-33A, miR-33B, miR-122, miR-370, miR-34c-5p, miR-351-5p, miR-378b, miR-186, miR-34a, miR-125b, miR-495, miR-766, miR-25, miR-338-3p, miR-92a-3p, miR-10396b-5p, miR-6787-5p, miR-1908-5p, miR-663a, miR-541-3p, and miR-654-5p.

70. The method of claim 64 wherein said at least two SIRT6-targeted miRNAs are selected from the group consisting of the miRNAs denoted by SEQ ID Nos: 1-4 and 28-44.

71. The method of claim 64 wherein said at least two SIRT6-targeted miRNAs are miR-33A and miR-122.

72. The method of claim 64 wherein said at least two antisense oligonucleotides comprise anti-miRs selected from the group consisting of anti-miR-33A, anti-miR-33B, anti-miR-122, anti-miR-370, anti-miR-34c-5p, anti-miR-351-5p, anti-miR-378b, anti-miR-186, anti-miR-34a, anti-miR-125b, anti-miR-495, anti-miR-766, anti-miR-25 anti-miR-338-3p, anti-miR-92a-3p, anti-miR-10396b-5p, anti-miR-6787-5p, anti-miR-1908-5p, anti-miR-663a, anti-miR-541-3p, and anti-miR-654-5p.

73. The method of claim 64 wherein said at least two antisense oligonucleotides comprise anti-miRs selected from the group consisting of the anti-miRs denoted by SEQ ID Nos: 5, 6 and 45-63.

74. The method of claim 75 wherein said at least two antisense oligonucleotides comprise SEQ ID NO: 5 and SEQ ID NO: 6 or an active fragment thereof.

75. The method of claim 65 wherein said antisense oligonucleotides are conjugated to a moiety.

76. The method of claim 75 wherein said moiety is a targeting moiety.

77. The method of claim 75 wherein said moiety is selected from a group consisting of N-acetylgalactosamine (GalNAc), cholesterol and vitamin E.

78. The method of claim 65 wherein said antisense oligonucleotides comprise chemically modified nucleic acids.

79. The method of claim 78 wherein said modified nucleic acids comprise 2′ O-methoxyethyl-RNA (MOE) or 2′-F modifications.

80. The method of claim 79 wherein said anti-miR-33 comprises the modified sequence
TmGmCfAfAfTfGfCfAfAfCmTmAmCfAfAfTfGfCfAmCm,
and wherein said anti-miR-122 comprises the modified sequence
AmCmAfAfAfCfAfCfCfAfTmTmGmUfCfAfCfAfCfUfCfCmAm
wherein m is a 2′ MOE modified base and f is a 2′F modified base.

81. A pharmaceutical composition comprising a combination of at least two antisense oligonucleotides which target at least two SIRT6-targeted miRNAs and a suitable carrier or excipient, wherein said antisense oligonucleotides cause partial inhibition of the silencing activity of said at least two SIRT6-targeted miRNAs.

82. A method for increasing SIRT6 mRNA level or SIRT6 protein level in a cell comprising contacting said cell with an effective amount of at least two antisense oligonucleotides which target at least two SIRT6-targeted miRNAs, and which partially inhibit the silencing activity of said at least two SIRT6-targeted miRNAs.

83. A method for increasing the amount of a target protein in a cell comprising contacting said cell with an effective amount of at least two anti-miRNAs, wherein said at least two anti-miRNAs target miRNAs which bind to the mRNA of said target protein, and wherein each of said at least two anti-miRNAs partially inhibits the silencing activity of said miRNAs.
